(19) United States
(12) Patent Application Publication
(10) Pub. No.: US 2006/0024242 A1
(43) Pub. Date: Feb. 2, 2006

(54) Solubilizing liquid of refractory element and producing method thereof

(76) Inventor: Seiri Oshiro, Naha (JP)

Correspondence Address: Patrick G. Burns;GREER, BURNS & CRAIN, LTD., Suite 2500, 300 South Wacker Dr., Chicago, IL, 60606, US

(21) Appl. No.: 11/233,273

(22) Filed: Sep. 22, 2005

(30) Foreign Application Priority Data
Apr. 19, 2004 (JP) 2004-122877
Apr. 12, 2005 (JP) 2005-114548

(51) Int. Cl. A61K 9/14; A61K 31/724; A61L 9/04; A61K 38/43

(52) U.S. Cl. 424/46; 514/58; 424/195.18; 424/539; 424/94.1

(57) ABSTRACT

A method for solubilizing in water a refractory element not soluble in water can be a method of adding a refractory element to material water obtained by solving a solubilizer, and finely grinding the refractory element using a homogenizer. However, the mechanical method of grinding using a homogenizer cannot evenly particulate the element, and it is difficult to obtain a translucent solubilizing liquid which is stable for a long period. When an ethanol solution obtained by solving a refractory element is mixed with material water, the ethanol solution is instantaneously dispersed, and the refractory element is particulated into colloidal particles. When a solubilizer solution obtained by solving a solubilizer in water is added to the colloidal solution and gently stirred, the particle is bound to the solubilizer to obtain a translucent solubilizing liquid which is stable for a long period.

### BACKGROUND OF THE INVENTION

1. Field of the Invention

The present invention relates to a method of easily solubilizing refractory elements such as fat-soluble elements of herbal medicines, pharmaceutical drugs, etc. which are not water-soluble or not easily soluble in water, and producing soft drinks, health foods, lotions, water-soluble drugs, a solution for an injection, etc.

2. Description of the Prior Art

In the current health-oriented age, water-soluble drugs of biological origin are sold as tea, soft drinks, health foods, or lotions. The water-soluble drugs are easily solved in boiling water, and result in translucent solutions.

On the other hand, there are trials for water-solubilizing refractory elements which are called fat-soluble elements, hydrophobic elements, or oleophilic elements, and are not water-soluble or not easily water-soluble, but it has been difficult to solubilize the refractory elements into translucent solutions.

Conventionally, as a method for solubilizing a refractory element, a refractory element is added to material water in which a solubilizer having an HLB value of 13 or higher is solved by an O/W type emulsifier, and a powerful stirring device such as a homomixer, a homogenizer, etc. mechanically grinds a refractory element into fine particles, thereby commonly realizing solubilization. Furthermore, cyclodextrin, which is not a solubilizer but functions as a solubilizer and is oleophilic inside and hydrophilic outside the cyclic structure, includes a finely ground refractory element, and is water-solubilized outside. A homogenizer can be a ultra-high speed rotation homogenizer, an ultra-high pressure homogenizer, an ultrasonic homogenizer, etc.

For example, the following patent document 1 (Japanese Published Patent Application No. 2003-147201) and patent document 2 (Japanese Unexamined Patent Publication No. Hei 11-9187 use an ultra-high pressure homogenizer as a method for finely dispersing a refractory element in water.

However, the mechanical and physical grinding method of the homogenizer, etc. is inefficient, requires a long time of several minutes to several tens of minutes, and generates uneven large and small particles. Therefore, when particulation cannot be perfectly performed, large and small particles are mixed, opaque and unstable emulsification is performed, and the resultant product is separated into water and a refractory element over a long period of time. Therefore, it has been difficult to obtain a stable and translucent solubilizing liquid. Additionally, there has been the problem that a prolonged stirring operation using a homogenizer can degenerate the solubilizing liquid by a chemical reaction due to a local temperature rise.

On the other hand, there have been chemical dispersing methods that do not use a mechanical grinding method using the above-mentioned homogenizer, etc. For example, according to the patent document 3 (Japanese Published Patent Application No. 2002-87972), as a method for solubilizing refractory propolis, a surfactant which is soluble in water and ethanol and has an HLB value of 11 or more and a polyethylene glycol chain is solved for solubilization in an alcohol-extracted solution of propolis.

The above-mentioned patent document 3 is explained below. In claim 1, a surfactant having a polyethylene glycol chain is used. In claim 2, a surfactant is a mixture of polyoxyethylene hardened ricinus oil and polysolvate 80. In claim 2, after solving a surfactant in an alcohol-extracted solution of propolis, the resultant substance is mixed with water. In claim 5, after removing the ethanol from the solution in claim 3, the resultant substance is mixed with water.

In the patent document 4 (Japanese Published Patent Application No. 2000-007584), fat-soluble drugs are solubilized by using amine oxides with fat-soluble drugs and 1-menthol and ethanol with a surfactant.

The patent document 4 is explained below. Claim 1 describes a method for solubilizing fat-soluble drugs by using amine oxides with fat-soluble drugs and 1-menthol and ethanol with a surfactant.

The solubilizers used in the above-mentioned patent documents 3 and 4 are verified below. According to the patent document 3, a surfactant having a polyethylene glycol chain is used, but such a surfactant having a polyethylene glycol chain is a suspected carcinogen, causing liver trouble, and kidney trouble when taken into the human body.

The solubilizing method according to the patent document 4 is to make fat-soluble drugs coexist with amine oxides, and to make a surfactant coexist with 1-menthol and ethanol. Amine oxides are recognized as hazardous substances in the Japanese PRTRR Law (Chemical Component Control Law). They cause problems when used in foods, cosmetics, and drugs.

As a finding in studying surfactants in the patent document 3, surfactants containing hydroxyl glycerin or polyglycerin are not useful because they cannot solve alcohol-extracted solutions, or be used as a solubilizer of safe glycerin fatty acid esters of natural origin.

Furthermore, relating to the propolis solubilizing method according to the patent document 3, claims 3 and 4 describe adding a surfactant to a propolis-extracted liquid and solving therein, and then mixing the resultant substance with water, but they have a defective point in that, as a result of an additional examination of the Applicant of the present invention, the propolis cannot be sufficiently solubilized when it is in high density.

### SUMMARY OF THE INVENTION

The present invention is a method of mixing an alcoholic solution with water, equally particulating a refractory element, and then binding an emulsifier to the resultant substance. Therefore, the mechanisms of the particulation and solubilization are quite different from each other.

The present invention aims at providing a method for solubilizing a refractory element not by a mechanical method using a homogenizer, but by a chemical method by immediately dispersing a refractory element in material water and evenly particulating the element, and then binding a solubilizer to particles to obtain a translucent solubilizing liquid, and a method for using sage elements for the human body.

The present invention is a device for solving the problem caused when a refractory element is particulated by a mechanical method using a homogenizer, etc. Alcohol is soluble in water, and when water is added to alcohol, the alcohol is evenly solved in the water. On the other hand, an alcoholic solution obtained by solving a refractory element in alcohol contains evenly dispersed alcohol in the water, and the refractory element in the solution is particulated into colloidal particles. When a solubilizer is added to the colloidal solution, a translucent solubilizing liquid can be obtained.

To be more specific, first, a refractory element is solved in alcohol to obtain an alcoholic solution. Similarly, a solubilizer is solved in water to obtain a solubilizer solution. Then, an appropriate amount of the above-mentioned alcoholic solution is added to material water containing only water or water type material such as tea, etc. (hereinafter referred to as material water) which functions as the base of a solubilizing liquid. The alcoholic solution is immediately dispersed in water, and the refractory element is evenly particulated into colloidal particles. While gently stirring the colloidal solution, the proper quantity of solubilizer solution is added to the colloidal solution. In this way a translucent solubilizing liquid can be obtained. This operation can be performed quickly and requires only a short amount of time.

According to the present invention, a mechanical method using a homogenizer, etc. is not required in particulating a refractory element but particulation is chemically performed. Additionally, the size of particles is uniform and very fine. According to other documents, the solution becomes semitransparent with a particle size of 200 nanometers or less, that is, less than or half the wavelength of visible light in diameter in the solubilizing liquid. This corresponds to the size of the colloidal particles of 1 through 100 nanometers. When particulation is performed according to the present invention, a completely translucent solution can be obtained. Therefore, it proves that the size is about 50 nanometers or less.

Furthermore, since the diameter of the hollow cyclodextrin is 0.6 through 1.0 nanometer, and the particles obtained as described above can be solubilized with the cyclodextrin positioned adjacent to or enclosed by the cyclodextrin, it proves that the size of the particles is smaller than the diameter of the hollow cyclodextrin of 1.0 nanometer.

The particle size can be adjusted by the density of the refractory element in the alcoholic solution. That is, the lower the density, the smaller the particles, thereby more easily realizing solubilization. Conversely, the higher the density, the larger the particles, thereby the less easily is solubilization realized and the more turbid an emulsifier is obtained. These findings indicate that the present invention is complete, and describe the features of the present invention.

There is a device for binding a solubilizer to the particles, and is described sequentially below. The first solving device is a method of mixing an alcoholic solution obtained by extracting or solving a refractory element in alcohol with material water, dispersing and particulating the refractory element, quickly adding a solubilizer solution to the material water, binding the particles to a solubilizer, and realizing solubilization. In this method, a refractory element is first dispersed in water and particulated, and then a solubilizer is bound to a particle for solubilization.

In this case, the solubilizer solution is added bit by bit, and the minimum amount of solubilizer can be used while adjusting the translucence.

The second solving device is to realize particulation by adding an alcoholic solution of a refractory element to material water in which a solubilizer is solved in advance based on the data obtained by the first device, and simultaneously binding a solubilizer to a particle. The obtained translucent solubilizing liquid is exactly as translucent as the solubilizing liquid obtained by the first solving device. In this case, when an alcoholic solution is added to material water, the portion where the alcoholic solution is added becomes instantaneously turbid by colloidal particles, but immediately returns to a translucent solution. This indicates that the alcoholic solution is dispersed in water, generates particles, and then is bound to the solubilizer.

The third solving device obtains a translucent solubilizing liquid containing a refractory element of high density in addition to a solubilizer solution in which the alcoholic solution is solved in a small amount of water. As necessary, an appropriate amount of solubilizing liquid is added to material water or a water type material such as tea, a soft drink, etc.

The fourth solving device is a method of producing non-alcohol solubilizing liquid by removing the largest amount possible of alcohol in a heating or decompressing method from the solubilizing liquid obtained by the first, second, and third solving devices. This method is used for soft drinks, cosmetics, drugs, etc. However, completely removing alcohol from a water solution has the problem of not being economical, and is used when only a small amount of alcohol residue can be accepted.

A stabilization check on the solubilizing liquid obtained by the first, second, third, and fourth solving device is performed as follows. The liquid is left in a room at the temperature of 10 through 25° C. for eight months. However, no solubilizing liquid indicates any change, and the solubilizing liquid remains completely translucent and stable without separation between water and the refractory element.

The alcohol according to the present invention is an alcohol soluble in water, solves a refractory element, and can be methanol, ethanol, 2-propanol, n-butyl alcohol, tertiary butanol, 1- and 4-buthylene glycol, 1- and 3-buthylene glycol, 2-ethyl hexyl alcohol, pentanol, etc. Considering the safety of the human body, it is preferable to use ethanol for pharmaceutical drugs and foods, and ethanol or 1- and 3-buthylene glycol (1,3 BG) for cosmetics, but the present invention is not limited to these applications.

The emulsifier according to the present invention is an O/W emulsifier having an HLB value of 8 or more, and emulsifies or solubilizes a particulated refractory element. If it is particulated in the production method according to the present invention, particles of the size corresponding to any O/W type emulsifier having an HLB value of 8 or more can be produced. The translucence depends on the HLB value. That is, the HLB values of 8 through 10 produce a turbid emulsified solution. The HLB values of 10 through 13 can produce a somewhat turbid solubilizing liquid. The HLB values of 13 or more can produce a completely translucent solubilizing liquid. A solubilizer has an HLB value of 10 or more in the emulsifiers, and makes a solubilizing liquid which is half translucent to completely translucent.

An emulsifier can be a recognized safe food additive in Japan such as glycerin, fatty acid esters, sucrose fatty acid esters, sorbitan fatty acid esters, propylene glycol fatty acid esters, polyoxyethylene sorbitan fatty acid esters, stearin lactic acid calcium, lecithins, etc. Among them, a solubilizer has an HLB value of 10 or more, and it is preferable that the HLB value is 13 or more. The solubilizer is not limited to what is described above, but any type of solubilizer can be used if the solubilizing effect and safety can be preserved.

Additionally, cyclodextrin (CD for short) can be used as a solubilizer. Cyclodextrin is 6 through 8 units of circularly bound dextrose the inside of which is oleophilic and contains a particulated oleophilic refractory element close to the inside of the ring, and the outside of which is hydrophilic and water-soluble, and functions as a solubilizer. The diameter of the hollow is 0.6 through 1.0 nanometer, the outer diameter is 1.0 through 2.0 nanometers, which is an appropriate size for solubilization. Cyclodextrin can be of α-CD, β-CD, and γ-CD types indicating water solubility's of 14.5% of an γ-CD type, 18.5% of a γ-CD type, and 23.2% of a γ-CD type. The γ-CD type is desired for solubilization.

The solubilizing liquid using the CD (cyclodextrin) described in the paragraph above is dried by a spray drier to produce CD powder containing a refractory element. The produced CD powder can be solved by a predetermined amount of water to produce a solubilizing liquid similar to the solubilizing liquid described in the paragraph above. The advantage of CD powder is that the powder can be used by decreasing the volume and weight as compared with a solubilizing liquid, can be used with improved preservation, and can be added to various foods. Spray drying can be performed in a common method. For example, by applying a spray of the solubilizing liquid described in the paragraph above by a disk rotating at a high speed in a spray drier into which heated air of about 170° C. is supplied, the water of the solubilizing liquid can be immediately evaporated, thereby producing the CD powder.

The refractory element according to the present invention is a fat-soluble element, a hydrophobic element, or an oleophilic element which is not soluble or not easily soluble in water, but is soluble or easily soluble in alcohol. To be specific, it can be: curcumin effective for liver trouble contained in the rootstalk of turmeric (Curcuma longa); capsaicin which is a spice element contained in hot pepper (Capsicum annuum) effective for adipose metabolism: paclitaxel which is an anticancer drug contained in the leaf of English yew (Taxus brevifolia); propolis of honey; coenzyme Q10 (commonly called ubidecarenone C59H9004) which is an element generated in the human body; corosolic acid contained in the leaf of jarool (Lagerstroemia speciosapers) effective for diabetes; tagitinin C contained in the leaf of tithonia (Thithonia diversifolia (Hemsl)) effective for liver trouble and diabetes; an alcohol-soluble component contained in the seed, bark, and leaf of azedarach (Melia azedarach L. or Milia azedarach L. var. subtripinnate miq.) effective as an anti-tumor drug; an alcohol-soluble component contained in the seed, bark, and leaf of neem (Azadiractha indica) containing the insect repellent used as agricultural chemicals in the past in Ayurveda, India, and the element effective against diabetes, anti-ulcer drug, anti-inflammation drug, etc.; an alcohol-soluble component such as damnakantar, etc. contained in the stalk and root of nonu (Morinda citrifolia) having an analgesic effect and an anti-allergy effect, etc. Furthermore, biological portions, cell contents, secreta, and so called extracted biological medicines, or chemical composite substances which can be foods, health foods, cosmetics, and drugs, or can be materials as is.

The above-mentioned elements can have a multiplier effect by combining two or more types of the elements, and it is desirable that a plurality of types are combined in developing a product.

The above-mentioned solubilizing liquid can be used for soft drinks, health drinks, lotions, liquid drugs, solutions for an injection, drops, etc. depending on the purpose. For example, for soft drinks and health drinks, the solubilizing liquid is provided with sweeteners, acidulants, perfumes, thickeners and stabilizers, colorants, preservatives, inhibitors, supplements, etc. depending on the purpose of the desired product in the market.

### DESCRIPTION OF THE PREFERRED EMBODIMENT

The embodiments of the present invention are described below by referring to the attached drawings.

The best mode for the present invention is explained below.

First a refractory element is extracted or solved by ethanol, thereby producing an ethanol solution. The glycerin fatty acid ester solubilizer having an HLB value of 16 is solved in water to produce a solubilizer solution. When an appropriate amount of the ethanol solution is mixed with the material water which is the base of the solubilizing liquid, the ethanol solution is dispersed in water, and the refractory element is evenly particulated into colloidal particles. An appropriate amount of the above-mentioned solubilizer solution is added to the colloidal particles and the mixed substance is gently stirred. Thus, a translucent solubilizing liquid can be obtained. This operation is performed immediately without taking a long time. The amount of the ethanol solution is 1% or less in the case of drinking water to be consumed in Japan.

Based on the usage of the solubilizer obtained above, the solubilizer solution is added to the material water. An appropriate amount of ethanol solution is added to the water while gently stirring the ethanol solution to obtain a translucent solubilizing liquid.

Depending on the density of the curcumin, the ethanol solution and the solubilizing liquid of curcumin which is a refractory element contained in the rootstalk of turmeric becomes yellow, orange, brown, etc., and is appropriate as an index element of solubilization. Therefore, it is used in the embodiment of the present invention.

The powder of the dry rootstalk of turmeric (Curcuma longa) containing curcumin in high density is extracted in ethanol to produce an ethanol solution containing curcumin. The glycerin fatty acid ester solubilizer having an appropriate HLB value of 16 is solved in the material water to produce a solubilizer solution. Then, the ethanol solution containing curcumin is added to the material water while gently stirring the solution to obtain a turbid yellow colloidal solution. While gently stirring the turbid yellow colloidal solution, an appropriate amount of solubilizer solution is added to the colloidal solution to obtain a yellow and translucent solubilizing liquid.

The tetrahydrocurcumin which is the biotransformation product of curcumin is a powerful anti-oxidant, and it is expected it will be used as an element as a lotion protecting the skin against harmful ultraviolet. In the embodiment, ethanol is replaced with 1,3 buthylene glycol (BG).

### Embodiment 1

0.7 g of the powder of dry rootstalk of turmeric containing 5.1% of curcumin is extracted in 200 ml of 95% ethanol at 70° C. for 6 hours to produce a deep brown ethanol solution containing curcumin. 1 g of the glycerin fatty acid ester solubilizer having an HLB value of 16.0 is solved in water to produce a solubilizer solution. Then, 100 ml of material water is added to 1 ml of the ethanol solution while gently stirring them to obtain a turbid yellow colloidal solution. While gently stirring the colloidal solution, 0.8 ml of the solubilizer solution is added to obtain a yellow and translucent solubilizing liquid.

### Embodiment 2

1.0 ml of solubilizer solution produced in the embodiment 1 is added to 100 ml of material water and solved. 1 ml of ethanol solution produced in the embodiment 1 is added to the material water, and the resultant solution is stirred to produce a yellow and turbid solubilizing liquid.

### Embodiment 3

0.5 g of 98% curcumin is solved in 100 ml of 95% ethanol to produce an ethanol solution. Then, 1 ml of ethanol solution is added to 100 ml of material water to produce a turbid yellow colloidal solution. 1 ml of the solubilizer solution produced in the embodiment 1 is added to the colloidal solution, and the solution is gently stirred to produce a yellow and translucent solubilizing liquid.

### Embodiment 4

5 ml of water is added to 1 ml of solubilizer solution produced in the embodiment 1, to which 1 ml of ethanol solution produced in the embodiment 1 is added and stirred to obtain a translucent solubilizing liquid containing a high density of curcumin. 7 ml of the obtained solubilizing liquid is added to 100 ml of commonly marketed tea to obtain tea containing curcumin.

### Embodiment 5

1 g of tetrahydrocurcumin is solved in 100 ml of 3 buthylene glycol to obtain a colorless transparent solution of 1,3 buthylene glycol. 1 ml of 1,3 buthylene glycol solution is added to 100 ml of material water to obtain a white and turbid colloidal solution, to which 1 ml of solubilizer solution produced in the embodiment 1 is added and gently stirred to obtain a water-clear lotion containing tetrahydrocurcumin.

### Embodiment 6

1 g of γ-CD (γ-cyclodextrin) is solved in 10 ml of water to produce a γ-CD solution. 1 ml of ethanol solution containing curcumin of the “embodiment 1” is added to 100 ml of material water to obtain a yellow and turbid colloidal solution, to which 1 ml of the γ-CD solution is added and gently stirred to obtain a yellow and translucent solubilizing liquid.

### Embodiment 7

1000 ml of solubilizing liquid obtained in the embodiment 6 is dried by the spray drier, and about 10 g of pale yellow CD powder containing a refractory element is obtained. When the obtained CD powder is added to 1000 ml of water and the solution is stirred, a clear yellow solubilizing liquid can be obtained.

The present invention has been developed by applying the phenomenon in which a refractory element mechanically particulated conventionally using homomixer or a homogenizer is instantaneously dispersed in an alcoholic solution. As compared with the homogenizer, etc. the present invention has the following merits.
1. It is not a physical or mechanical process, but a chemically process.
2. The particle size is 200 nanometers or less.
3. The dispersion and particulation in water are instantaneously performed, and do not require a long time.
4. Since the particulation is instantaneously performed, oxidation can be suppressed.
5. Since a homogenizer mechanically stirs a solution at high speed, partial shearing or high temperatures occur, thereby readily causing chemical degradation. However, the present invention is free of this demerit.
6. An obtained solubilizing liquid is translucent for a long period and stable water and a refractory element not separating.
7. The homogenizer throughput depends on the size of the machine, but the present invention can perform a process within a short time regardless of the amount of process.
8. There are an enormous number of elements hardly or not solved in water, but solved in alcohol. They can be solubilized.
9. A homogenizer which can particulate is expensive and requires resources, however the present invention is economical.
10. Since a solubilizer can be added while checking the translucence of material water, the amount of solubilizer to be added can be reduced to the least possible amount.

1. A solubilizing liquid of a refractory element, comprising:
a device dispersing an alcoholic solution obtained by solving the refractory element material in water, and particulating the refractory element; and
a device binding the particle to a emulsifier.

2. The solubilizing liquid of the refractory element according to claim 1, wherein the refractory element includes one or more types selected from among alcohol-soluble components contained in curcumin, tetrahydrocurcumin, capsaicin, paclitaxel, propolis, coenzyme Q10, corosolic acid, tagitinin, and azedarach, an alcohol-soluble component contained in neem, and an alcohol-soluble component contained in the root and stalk of nonu.

3. The solubilizing liquid of the refractory element according to any of claims 1 or 2, wherein the alcohol is selected from among methanol, ethanol, 2-propanol, n-butyl alcohol, tertiary butanol, 1,4-buthylene glycol, 1,3-buthylene glycol, 2-ethyl hexyl alcohol, and pentanol.

4. The solubilizing liquid of the refractory element according to any of claims 1 or 2, wherein a size of the particle is 200 nanometers or less.

5. The solubilizing liquid of a refractory element according to any of claims 1 or 2, wherein the emulsifier is selected from among glycerin fatty acid esters, sucrose fatty acid esters, sorbitan fatty acid esters, propylene glycol fatty acid esters, polyoxyethylene sorbitan fatty acid esters, stearin lactic acid calcium, and lecithins, and has an HLB value of 10 or more.

6. The solubilizing liquid according to any of claims 1 or 2, wherein the emulsifier is cyclodextrin.

7. A method of producing a solubilizing liquid of a refractory element, comprising:
a process of dispersing an alcoholic solution obtained by solving the refractory element material in water, and particulating the refractory element; and
a process of binding the particle to a emulsifier.

8. The method of producing a solubilizing liquid of the refractory element according to claim 7, wherein the refractory element includes one or more types selected from among alcohol-soluble components contained in curcumin, tetrahydrocurcumin, capsaicin, paclitaxel, propolis, coenzyme Q10, corosolic acid, tagitinin, and azedarach, an alcohol-soluble component contained in neem, and an alcohol-soluble component contained in the root and stalk of nonu.

9. The method of producing a solubilizing liquid of the refractory element according to any of claims 7 or 8, wherein the alcohol is selected from among methanol, ethanol, 2-propanol, n-butyl alcohol, tertiary butanol, 1,4-buthylene glycol, 1,3-buthylene glycol, 2-ethyl hexyl alcohol, and pentanol.

10. The method of producing a solubilizing liquid of the refractory element according to any of claims 7 or 8, wherein a size of the particle is 200 nanometers or less.

11. The method of producing a solubilizing liquid of a refractory element according to any of claims 7 or 8, wherein the emulsifier is selected from among glycerin fatty acid esters, sucrose fatty acid esters, sorbitan fatty acid esters, propylene glycol fatty acid esters, polyoxyethylene sorbitan fatty acid esters, stearin lactic acid calcium, and lecithins, and has an HLB value of 10 or more.

12. The method of producing a solubilizing liquid according to any of claims 7 or 8, wherein the emulsifier is cyclodextrin.

13. A dry CD powder containing the refractory element according to claim 6, wherein the solubilizing liquid can be dried.

14. A method of producing a CD dry powder containing a refractory element, wherein the solubilizing liquid obtained in the producing method according to claim 12 is further dried.
